(19) United States
(12) Patent Application Publication
(10) Pub. No.: US 2006/0216359 A1
(43) Pub. Date: Sep. 28, 2006

(54) Topical preparation for treating ulcerations on the inside of the month

(76) Inventor: Lowell Riegel, Carmi, IL (US)

Correspondence Address: Gary K. Price, Esq.;Bowers Harrison, LLP, 25 N.W. Riverside Drive, P.O. Box 1287, Evansville, IN, 47706-1287, US

(21) Appl. No.: 11/389,424

(22) Filed: Mar. 24, 2006

Related U.S. Application Data
(60) Provisional application No. 60/664,775, filed on Mar. 25, 2005.

(51) Int. Cl. A61K 33/40; A61K 36/534; A61K 31/05

(52) U.S. Cl. 424/616; 424/747; 514/731

(57) ABSTRACT

A topical preparation for treating ulcerations on the inside of the mouth. The composition includes initial preparation of a first mixture by adding glycerin and liquid phenol to water, preparation of a second mixture by adding glycerin and a flavoring agent such as peppermint oil to water, then, blending the first and second mixtures with hydrogen peroxide. The topical pharmaceutical formulation of this invention is applied directly to the canker sore.

### CROSS REFERENCES TO RELATED APPLICATIONS

U.S. Provisional Application for Patent No. 60/664,775, filed Mar. 25, 2005, with title “Topical Preparation for Treating Ulcerations on the Inside of the Mouth” which is hereby incorporated by reference. Applicant claims priority pursuant to 35 U.S.C. Par. 119(e)(i).

Statement as to Rights to inventions made under Federally sponsored research and development: Not applicable.

### BACKGROUND OF THE INVENTION

1. Field of the Invention.

This present invention relates to a method and composition for treating ulcerations inside the mouth commonly known as canker sores. More particularly, the invention relates to a method for treatment of canker sores using a homogeneous composition having as a basis the protective properties of phenol. Most particularly, the invention relates to a treatment of canker sores inside the mouth using a topically administered homogeneous composition directly to the lesion.

2. Background Information

Ulcerations inside the mouth, often referred to as canker sores is likely the most common disorder of the mouth, causing discomfort and annoyance to millions of Americans. “Canker sores” as used herein occur only inside the mouth, including on the tongue and the inside linings of the cheeks, lips and throat. Canker sores (Recurrent Aphthous Stomatitis, Aphthous Ulcers, Oral Ulcers) are mouth sores which plague millions of people. For some, canker sores are a minor nuisance and for others are constant sources of pain and discomfort. It appears that diet may influence episodes of canker sores as some people have allergies to certain foods and consumption of these foods can lead to canker sores. Recurrent canker sores afflict about twenty percent (20%) of the general population and are usually found of the movable parts of the mouth such as the tongue or the inside linings of the lips and cheeks. Without medication, it is not uncommon for such a sore to remain in excess of one week and take two weeks or longer to completely heal.

Usually a person will have only one or a few canker sores at a time. Most people have their first bout with canker sores between the ages of 10 and 20, but children as young as two may develop the condition. The frequency of canker sore recurrences varies considerably, some people have only one or two episodes a year while others may have a continuous series of canker sores.

There are several treatments for reducing the pain of canker sores for patients whose outbreaks cannot be prevented. These include numbing ointments such as benzocaine, which are available in drug stores without a prescription. Anti-inflammatory steroid mouth rinses or gels can be prescribed for patients with severe sores. Mouth rinses containing the antibiotic tetracycline may reduce the unpleasant symptoms of canker sores by preventing bacterial infections in the sores. The U.S. Food and Drug Administration warns, however, that tetracycline given to pregnant women and young children can permanently stain youngsters' teeth. Both steroid and tetracycline treatments require a prescription and care of a dentist or physician. Patients with severe recurrent canker sores may need to take steroid or other immunosuppressant drugs orally. These potent drugs can cause many undesirable side effects, and should be used only under the close supervision of a dentist or physician.

There are also a number of over-the-counter medications for such ulcerations including BLISTEX, ZILACTIN, and CAMPHO PHENIQUE. However, for many persons suffering from ulcerations inside the mouth, none of these medications is very effective. While these medications may assist in temporarily relieving the pain and discomfort associated with such ulcerations, they are not a cure. As a result, while known medication may temporarily relieve some of the discomfort caused by the sore, the sore will remain and run its normal standard of time to heal itself of approximately two weeks as discussed above. In contrast thereto, the composition which is the subject of the present invention has been found to bring relief and to effect healing much more quickly.

Prior art medications act primarily as an antiseptic and as an anesthetic and not as a cure, while the ulceration heals at its normal standard of time. The composition of this invention is an effective cure.

Accordingly, there is a need for an improved medication that not only relieves the pain and discomfort associated with ulcerations on the inside of the mouth, but also provides an effective cure in order to substantially reduce the normal standard of healing time. The present invention meets this need.

### SUMMARY OF THE INVENTION

According to the present invention, a novel method and composition is provided for treating ulcerations on the inside of the mouth which requires topically administering the composition directly to the lesion. The composition comprising initial preparation of a first mixture by adding glycerin and phenol to water, preparation of a second mixture by adding glycerin and a flavoring such as peppermint oil to water, then, blending the first and second mixtures with hydrogen peroxide.

Glycerin

Glycerin is highly “hygroscopic” which means that it absorbs water from the air. Diluted with water, glycerin will soften your skin and acts as a moisturizer and may have a soothing effect upon the affected area.

Phenol

Phenol is often used as a disinfectant, as an antiseptic, and is often used in medicinal preparations, such as mouthwash and sore throat lozenges.

Peppermint Oil

Peppermint oil is used for colds, flu, sore throat, laryngitis, and mild digestive disorders. Peppermint oil is one of the most extensively used in all the volatile oils, both medicinally and commercially. In the present invention, peppermint oil is used as a flavoring agent however, as discussed, peppermint oil further includes medicinal benefits.

Hydrogen Peroxide

Hydrogen peroxide is a clean, colorless liquid used in the present application as a disinfectant. The oxidizing power of hydrogen peroxide has been used as a treatment for minor cuts and scratches. Its oxidizing properties are effective in inhibiting growth and killing microbes. What makes hydrogen peroxide unique is the foaming action that takes place whenever it is placed on a lesion. A benefit of that foaming action is that hydrogen peroxide acts as a cleaning agent. The present invention is topically administered directly to the lesion. The composition not only relieves the pain and discomfort associated with canker sores, but also provides an effective cure in order to substantially reduce the normal standard of healing time.

### BRIEF DESCRIPTION OF THE DRAWINGS

FIG. 1 is a flow chart illustrating a preferred overall procedure for making a composition for treating ulcerations on the inside of the mouth, in accordance with the present invention.

### DESCRIPTION OF THE PREFERRED EMBODIMENTS

In accordance with the present invention, a method and composition for treating ulcerations inside the mouth that are often referred to as canker sores is disclosed. The method and composition is directed to a medication that is topically administered to the lesion. In the preferred embodiment, the composition of the present invention not only relieves the pain and discomfort associated with canker sores, but also provides an effective cure in order to substantially reduce the normal standard of healing time. In the broadest context, the composition consists of components configured and correlated with respect to each other so as to attain the desired objective.

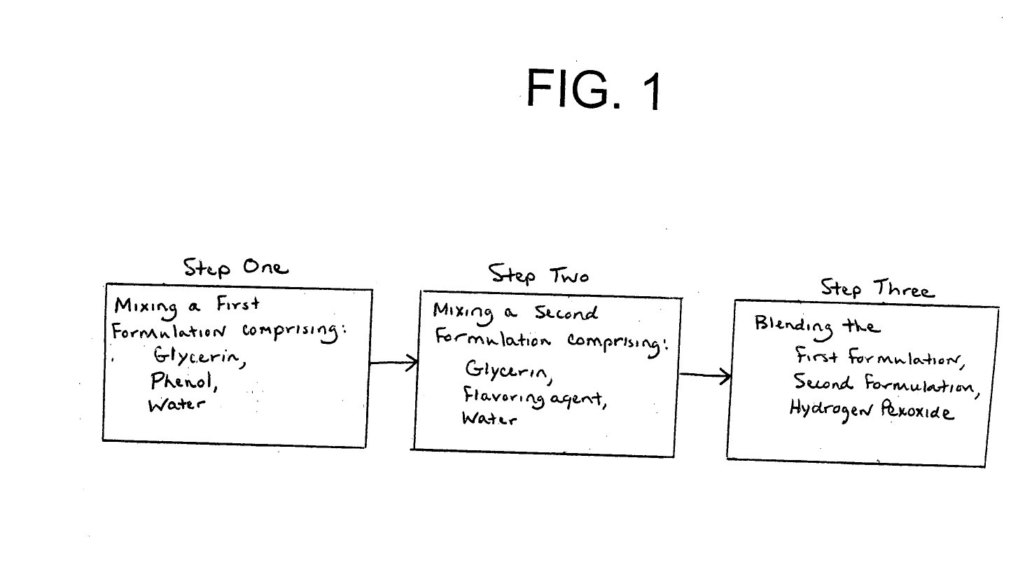

FIG. 1 is a flow chart illustrating a preferred overall procedure for making a composition for treating ulcerations on the inside of the mouth, in accordance with the present invention.

The formulation as will be described, is for total mixture equal to 100 ml to be blended with an equal volume of hydrogen peroxide to form the topical treatment composition of the present invention, and the volume for each component will be disclosed in milliliters (ml). Generally, the topical treatment composition involves preparation of a first formulation then a second formulation and then blending the first and second mixtures with hydrogen peroxide as follows:

Formulation 1

| | |
|---|---|
| glycerin | approx. 0.9% ml |
| phenol | approx. 1.8% ml |
| water | approx. 47.3% ml |

Formulation 2

| | |
|---|---|
| glycerin | approx. 1.0% ml |
| water | approx. 49% ml |
| flavoring | approx. 2 drops |

Topical Treatment

| | |
|---|---|
| hydrogen peroxide | approx. 100 ml or volume equal to the combined |
| | volume of Formulations 1 and 2 |

The mixing steps of the topical treatment composition are as follows:

First Step

The first step in preparing the composition of the present invention includes initial preparation of a first mixture by adding approximately 0.9 ml of glycerin and approximately 1.8 ml of liquid phenol to approximately 48 ml of water. Glycerin is known for its properties as a topical moisturizer when diluted with water, and serves to reduce the pain associated with the canker sore lesions and may have a soothing effect upon the lesions while the phenol acts to cause the lesions to heal. The mixture is then blended by ordinary means known in the art forming a first formulation.

While a pharmaceutical formulation in accordance with the present invention can vary, Applicant presently contemplates that the first formulation will comprise as discussed above namely, at least 0.9 ml of glycerin and approximately 1.8 ml of phenol to approximately 48 ml of water.

Second Step

The second step in preparing the composition of the present invention includes preparation of a second mixture by adding approximately 1.0 ml of glycerin and a selected amount, preferably approximately 2 drops of flavoring, such flavoring preferably peppermint oil, to approximately 49 ml of water. Peppermint oil, in the preferred embodiment, is used primarily as a flavoring agent for the formulation. Peppermint oil, however, is used medicinally for colds, flu, sore throat, laryngitis, and mild digestive disorders. As such, while peppermint oil provides the preferred flavoring oil, it may be added to the pharmaceutical formulation to enhance its medicinal characteristics. This mixture is then blended by ordinary means known in the art forming a second formulation.

While the pharmaceutical formulation in accordance with the present invention can vary, Applicant presently contemplates that the second formulation will comprise as discussed above namely, at least 1.0 ml of glycerin and selected amount of volatile oils, preferably peppermint oil, to approximately 48 ml of water.

Again, while the preferred embodiment uses peppermint oil as a flavoring, other flavoring agents known in the art can be used as a replacement.

Third Step

The third step in preparing the composition of the present invention includes blending the first formulation and second formulation with approximately equal parts of hydrogen peroxide until a homogeneous topical treatment composition is obtained. Hydrogen peroxide is known for its properties as a topical disinfectant and serves as a cleaning agent while the selected components of the first and second formulations act to cause the lesions to heal. This homogeneous composition then can be applied topically to the lesions, as previously described.

While a pharmaceutical formulation in accordance with the present invention can vary, Applicant presently contemplates that the formulation comprising of approximately equal parts of hydrogen peroxide to the total volume of the blended first formulation and second formulations is preferable.

Applied topically, the pharmaceutical formulation of the present invention must be brought into direct contact with the affected area. As such, the topical pharmaceutical formulation of this invention should be applied directly to the canker sore. Normally, topical application of the formulation should begin at the first signs of a lesion and continue until the lesion is completely eradicated. Although therapy should begin as early as possible, Applicant has found that topical treatment of fully erupted canker sores will enhance healing and thus believes that the formulation is effective when applied at any stage of lesion development. Application of the pharmaceutical formulation of the present invention has yielded very good results with no reported adverse side effects.

As many different embodiments of this invention will now occur to those skilled in the art, it is to be understood that the specific embodiments of the invention as presented herein are intended by way of illustration only and are not limiting on the invention. As such, while the description above contains many specificities, these should not be construed as limiting the scope of the invention but as merely providing illustrations of some of the presently preferred embodiments of this invention. It is to be understood that the present invention is not limited to the embodiments described above, but encompasses any and all embodiments within the scope of the claims.

It will be obvious to those skilled in the art that modifications may be made to the embodiments described above without departing from the scope of the invention. Thus the scope of the invention should be determined by the claims in the formal application and their legal equivalents, rather than by the examples given.

I claim:

1. A pharmaceutical composition for topical treatment of canker sores consisting essentially of:
a composition mixed by blending a total of approximately 1.9 ml of glycerin, a total of approximately 1.8% ml of liquid phenol, a total of approximately 96.3 ml of water, and approximately 2 drops of a flavoring agent, said composition having a volume, and
a final composition mixed by blending said composition with an equal volume of hydrogen peroxide.

2. The pharmaceutical composition as recited in claim 1, wherein the flavoring agent is peppermint oil.

3. A method of treating canker sore lesions comprising the following steps in the sequence set forth:
blending approximately 0.9 ml of glycerin, and approximately 1.8 ml of water phenol with approximately 47.3 ml of water to form a first formulation;
blending approximately 1.0 ml of glycerin with approximately 49 ml of water to form a second formulation;
adding approximately 2 drops of a flavoring agent to said second formulation;
blending said first formulation and said second formulation with an equal volume of hydrogen peroxide; and
topically applying the resulting composition to the lesions.

4. The method as recited in claim 3, wherein the flavoring agent is peppermint oil.
